(19) United States
(12) Patent Application Publication
(10) Pub. No.: US 2026/0344621 A1
(43) Pub. Date: Nov. 5, 2026

(54) CHEMICAL DISBUDDING METHOD

(71) Applicant: WELFARE CONCEPTS LIMITED, Golflands, Auckland (NZ)

(72) Inventors: John Paul CHAMBERS, Golflands, Auckland (NZ); Preet Mohinder SINGH, Golflands, Auckland (NZ); Richard Gerhardus Martinus OLDE RIEKERINK, Golflands, Auckland (NZ); Forbes Richard EMSLIE, Golflands, Auckland (NZ); Ian George TUCKER, Golflands, Auckland (NZ); Dinakaran VENKATACHALAM, Golflands, Auckland (NZ)

(21) Appl. No.: 19/108,088

(22) PCT Filed: Aug. 31, 2023
(86) PCT No.: PCT/IB2023/058595

§ 371 Date: Feb. 28, 2025

(30) Foreign Application Priority Data
Sep. 5, 2022 (AU) 2022902547

(51) Int. Cl. A01K 17/00

(52) U.S. Cl. CPC A01K 17/00

(57) ABSTRACT

A cytotoxic agent and its use in animal disbudding or for inhibiting horn growth in a juvenile animal. The cytotoxic agent can be a sclerosing agent, p53 activating agent, antiprotozoal agent, apoptosis inducing agent, or a local anaesthetic agent.

### RELATED APPLICATIONS

This application claims priority of Australian Provisional Patent Application No. 2022902547, filed 5 Sep. 2022, the entire contents of which are incorporated herein by reference.

### TECHNICAL FIELD

This invention broadly relates to a cytotoxic agent and its use in animal disbudding or for inhibiting horn growth in a juvenile animal. In particular, the cytotoxic agent is a sclerosing agent, p53 activating agent, antiprotozoal agent, apoptosis inducing agent, and/or a local anaesthetic agent.

### BACKGROUND

Horn disbudding is a routine husbandry practice in most dairy herds around the world.

Disbudding is a procedure in which the horn buds of a calf are removed or destroyed with the use of a thermal cautery (hot iron) unit to burn and destroy the tissue around the horn bud. The hot iron equipment used for disbudding is potentially hazardous due to the use of high temperatures. Successful disbudding requires experience and skill, while improper technique can lead to injuries to the animal and operator.

Even when administered correctly, a hot iron disbudding procedure will cause pain and distress to the animal if performed without appropriate anaesthesia and analgesia. In New Zealand, it is illegal to disbud a calf unless it is “under the influence of an appropriately placed and effective local anaesthetic” (Animal Welfare (Care and Procedures) Regulations 2018). The ideal welfare protocol for disbudding includes administration of a sedative to reduce the stress during the procedure, injection of local anaesthetic to the corneal nerve to anaesthetise the horn buds and surrounding tissue, and administration of a non-steroidal anti-inflammatory drug (NSAID) to reduce post-operative pain. However, the combination of these procedures is still not guaranteed to provide a comfortable experience for the animal. The cost and availability of the appropriate drugs to the animal handlers are hurdles in most countries. The recommended treatment as above also requires experience and skill in administering the proper drug regime. In particular, correctly administering an anaesthetic to the corneal nerve requires training on the proper procedure.

Other less common methods of disbudding include directly cutting out the horn bud with a scoop or gouge, and chemical disbudding using caustic soda. Removal of horn buds by cutting has been observed to cause more pain and distress than the use of a hot iron and leaves a large, open wound.

There are reports in literature of experimental methods of disbudding that have been trialed. A paper in 1976 (Koger L M. Dehorning by injection of calcium chloride. Vet Med Small Anim Clin 1976; 71 (6): 824-825) describes a method in which high concentrations of calcium chloride were injected into the horn bud, causing necrosis of the tissue. This method also caused significant inflammation and would be expected to cause considerable pain. The authors recommended the use of analgesics and sedatives.

The use of caustic soda has been trialed. Caustic soda often inadvertently spreads from the administration site to other areas of the animal and other animals through contact, can lead to blindness of the treated animals and is adversely affected by rain.

The use of liquid nitrogen as a cryogenic fluid to destroy the horn bud tissue has been attempted, but found to be almost entirely ineffective.

The injection of clove oil (eugenol) under the horn bud to cause necrosis of horn-bud tissue has been trialed, but was found to be relatively ineffective, delaying rather preventing horn growth in many cases, and was not recommended by the original investigators (Sutherland et al Vet Sci. 2019 December; 6 (4): 102).

Consequentially, there is a need for an improved composition or method for preventing the growth of horns in juvenile animals.

### OBJECT OF THE INVENTION

It is an object of one or more embodiments of the present invention to address one or more of the foregoing problems. Alternatively, it is an object of one or more embodiments of the present invention to provide a composition for, or method of, chemical disbudding that overcomes or minimises a disadvantage of known dehorning methods referred to above. Alternatively, it is an object of the present invention to provide the public with a useful choice.

### DETAILED DESCRIPTION OF THE INVENTION

The invention broadly relates to a cytotoxic agent and its use in animal disbudding.

According to a first aspect of the present invention, there is provided a method of chemically disbudding a juvenile animal by administering to horn bud cells of the animal at least one cytotoxic agent, wherein the at least one cytotoxic agent comprises at least one sclerosing agent, p53 activating agent, antiprotozoal agent, apoptosis inducing agent, or local anaesthetic agent.

According to a second aspect of the present invention, there is provided use of at least one cytotoxic agent for chemically disbudding a juvenile animal, wherein the at least one cytotoxic agent cytotoxic agent comprises at least one sclerosing agent, p53 activating agent, antiprotozoal agent, apoptosis inducing agent, or local anaesthetic agent.

According to a third aspect of the present invention, there is provided at least one cytotoxic agent for use or when used for chemically disbudding a juvenile animal, wherein the at least one cytotoxic agent comprises at least one sclerosing agent, p53 activating agent, antiprotozoal agent, apoptosis inducing agent, or local anaesthetic agent.

According to a fourth aspect of the present invention, there is provided use of at least one cytotoxic agent in the manufacture of a medicament for chemically disbudding a juvenile animal, wherein the at least one cytotoxic agent comprises at least one sclerosing agent, p53 activating agent, antiprotozoal agent, apoptosis inducing agent, or local anaesthetic agent.

According to a fifth aspect of the present invention, there is provided a disbudding composition comprising at least one cytotoxic agent, formulated for administration to and for chemically disbudding a juvenile animal, wherein the at least one cytotoxic agent comprises at least one sclerosing agent, p53 activating agent, antiprotozoal agent, apoptosis inducing agent, or local anaesthetic agent.

According to a sixth aspect of the present invention, there is provided a method of inhibiting horn growth in a juvenile animal by administering to horn bud cells of the animal at least one cytotoxic agent, wherein the at least one cytotoxic agent comprises at least one sclerosing agent, p53 activating agent, antiprotozoal agent, apoptosis inducing agent, or local anaesthetic agent.

According to a seventh aspect of the present invention, there is provided use of at least one cytotoxic agent for inhibiting horn growth in a juvenile animal, wherein the at least one cytotoxic agent comprises at least one sclerosing agent, p53 activating agent, antiprotozoal agent, apoptosis inducing agent, or local anaesthetic agent.

According to an eighth aspect of the present invention, there is provided at least one cytotoxic agent for use or when used for inhibiting horn growth a juvenile animal, wherein the at least one cytotoxic agent comprises at least one sclerosing agent, p53 activating agent, antiprotozoal agent, apoptosis inducing agent, or local anaesthetic agent.

According to a ninth aspect of the present invention, there is provided use of at least one cytotoxic agent in the manufacture of a medicament for inhibiting horn growth in a juvenile animal, wherein the at least one cytotoxic agent comprises at least one sclerosing agent, p53 activating agent, antiprotozoal agent, apoptosis inducing agent, or local anaesthetic agent.

According to a tenth aspect of the present invention, there is provided a composition comprising at least one cytotoxic agent, formulated for administration to and for inhibiting horn growth in a juvenile animal, wherein the at least one cytotoxic agent comprises at least one sclerosing agent, p53 activating agent, antiprotozoal agent, apoptosis inducing agent, or local anaesthetic agent.

According to an eleventh aspect of the present invention, there is provided an injector containing at least one cytotoxic agent for use or when used for juvenile animal disbudding or for inhibiting horn growth in a juvenile animal, wherein the injector is capable of administering the at least one cytotoxic agent to horn bud cells of a juvenile animal, wherein the at least one cytotoxic agent comprises at least one sclerosing agent, p53 activating agent, antiprotozoal agent, apoptosis inducing agent, or local anaesthetic agent.

According to a twelfth aspect of the present invention, there is provided a kitset for use or when used in a method of juvenile animal disbudding or inhibiting horn growth in a juvenile animal, wherein the kitset comprises: an injector capable of administering at least one cytotoxic agent to horn bud cells of a juvenile animal; and, the at least one cytotoxic agent in an amount to kill horn bud cells, wherein the at least one cytotoxic agent comprises at least one sclerosing agent, p53 activating agent, antiprotozoal agent, apoptosis inducing agent, or local anaesthetic agent.

According to a thirteenth aspect of the present invention, there is provided a composition formulated as a solution, suspension, dispersion, emulsion, low viscosity gel, or other prolonged-release liquid formulation, comprising at least one cytotoxic agent and one or more excipients, and being capable of providing prolonged-release of the at least one cytotoxic agent, wherein the at least one cytotoxic agent comprises at least one sclerosing agent, p53 activating agent, antiprotozoal agent, apoptosis inducing agent, or local anaesthetic agent.

According to a fourteenth aspect of the present invention, there is provided a composition formulated as a solution, suspension, dispersion, emulsion, low viscosity gel, or other prolonged-release formulation, comprising at least one cytotoxic agent in an amount capable of juvenile animal disbudding or inhibiting horn growth in a juvenile animal and one or more excipients, and being capable of providing prolonged-release of the at least one cytotoxic agent, wherein the at least one cytotoxic agent comprises at least one sclerosing agent, p53 activating agent, antiprotozoal agent, apoptosis inducing agent, or local anaesthetic agent.

According to a fifteenth aspect of the present invention, there is provided a composition formulated as a low viscosity, injectable gel comprising, all in approximate % w/V:
10% w/v of at least one cytotoxic agent, preferably mepacrine or a salt thereof that provides 10% w/v mepacrine, such as 11.8% w/v mepacrine dihydrochloride, or mepacrine dihydrochloride dihydrate;
40-85% w/v SAIB;
0-2% w/v Tween 80;
0-50% w/v Triacetin;
0-50% w/v DEGEE (Transcutol™); and
ethanol to 100% by volume, wherein:
the concentration of ethanol varies from 0-30% w/v; or
in the absence of other solvents and Tween, and at a SAIB level of 85% w/v, the ethanol concentration is approximately 15% w/v, assuming a weight per mL of the composition's formulation is 1.1 g.

According to a sixteenth aspect of the present invention, there is provided a composition formulated as a low viscosity, injectable gel comprising, all in approximate % w/v:
20% w/v of the at least one cytotoxic agent, preferably mepacrine or a salt thereof that provides 20% w/v mepacrine, such as 23.6% w/v mepacrine dihydrochloride, or 25.4% w/v mepacrine dihydrochloride dihydrate;
40-85% w/v SAIB;
0-2% w/v Tween 80;
0-50% w/v Triacetin;
0-50% w/v DEGEE (Transcutol™); and
ethanol to 100% by volume, wherein:
the concentration of ethanol varies from 0-30% w/v; or
in the absence of other solvents and Tween, and at 20% w/v mepacrine base and a SAIB level of 85% w/v, the ethanol concentration is 5% w/v, assuming a weight per mL of the composition's formulation is 1.1 g, which may be too low in which case the maximum concentration of SAIB would have to be reduced.

According to a seventeenth aspect of the present invention, there is provided a composition formulated as a low viscosity, injectable gel comprising, all in approximate % w/v:
10% w/v of the at least one cytotoxic agent, preferably mepacrine or a salt thereof that provides 10% w/v mepacrine, such as 11.8% w/v mepacrine dihydrochloride;
70% w/v SAIB;
1% w/v Tween 80;
5% w/v Triacetin;
0% w/v DEGEE (Transcutol™); and
ethanol to 100% by volume, that is about 22% w/v of ethanol assuming a weight per mL of the final formulation of 1.1 g and assuming mepacrine dihydrochloride is used.

According to an eighteenth aspect of the present invention, there is provided a composition formulated as a low viscosity, injectable gel comprising, all in approximate % w/v:
20% w/v of the at least one cytotoxic agent, preferably mepacrine, more preferably 23.6% w/v mepacrine dihydrochloride;
70% w/v SAIB;
1% w/v Tween 80;
5% w/v Triacetin;
0% w/v DEGEE (Transcutol™); and
ethanol to 100% by volume, that is about 10.4% w/v ethanol assuming a weight per mL of the final formulation of 1.1 g.

According to a nineteenth aspect of the present invention, there is provided a composition formulated as an injectable oily solution or suspension comprising:
10 to 15% w/v of at least one cytotoxic agent, preferably mepacrine or a salt thereof that provides 10 to 15% w/v mepacrine, such as 11.8 to 17.7% w/v mepacrine dihydrochloride; and
at least one oily excipient.

It is to be appreciated that features recited for a particular aspect of the present invention may be features, or recast as features, of any and all other aspects of the present invention, context permitting. For example, one or more features of a ‘method’ (method, use, process etc.) may be recast as features of a ‘product’ (cytotoxic agent, composition, medicament, formulation, injector, kitset etc.), and vice-versa, context permitting.

A ‘cytotoxic agent’ refers to an agent that is toxic to horn bud cells, particularly corium, which can be destroyed and/or inactivated by the cytotoxic agent. Unless stated otherwise, a ‘cytotoxic agent’ refers to at least one type of agent, which in some embodiments may be two, three, four or more different types of agents used together. For example, a further suitable cytotoxic agent may be eugenol (clove oil). Eugenol may be used, for example, in combination with mepacrine.

In some embodiments, the cytotoxic agent can be in a free-acid or free-base form, or a salt. Context permitting, the term ‘cytotoxic agent’ includes the free-acid or free-base form of the cytotoxic agent as well as any salt or salts of that cytotoxic agent. For example, mepacrine is a base/free-base, so use of ‘mepacrine’ throughout the description and claims includes mepacrine as a free-base as well as any salt of mepacrine, such as mepacrine dihydrochloride or mepacrine dihydrochloride dihydrate. Sometimes one or more salts of mepacrine may be specifically mentioned for greater clarity.

Although not always explicitly stated, it is to be appreciated that the cytotoxic agent will typically be administered to the animal in the form of a composition, formulation or medicament.

It is to be appreciated that disbudding or inhibition of horn growth can be achieved by administering the cytotoxic agent directly to horn bud cells (horn bud tissue) of the animal, or in an immediate vicinity of horn bud cells. Preferably, disbudding or horn inhibition is carried out whilst the horn bud is free-floating in the skin. Preferably disbudding or horn inhibition is carried out before the horn bud has attached to the skull. Preferably the cytotoxic agent is administered under or around the horn buds, more preferably under the horn buds. Successful disbudding or horn inhibition requires inactivation or destruction of horn-producing cells (corium) (ie. ‘horn bud cells’) of the horn bud.

Preferably the cytotoxic agent is administered in an amount (ie. an ‘effective amount’) sufficient to prevent horn growth, or significantly reduce horn growth such that the resulting horn/scur/stump/bud is not of danger to other animals. Preferably horn growth is prevented completely.

The inventors have found that disbudding of a juvenile animal can be performed by administering at least one (one or more) sclerosing agents to a horn bud.

Any suitable sclerosing agent or agents can be used. Techniques are known in the art for identifying and evaluating new potential sclerosing agents, including in vitro assays. Sclerosing agents are typically used in sclerotherapy. In a sclerotherapy procedure, a sclerosing agent generally refers to a substance that when introduced in the lumen of a vessel causes damage to the vessel wall, leading to occlusion of the vessel from resultant fibrosis. Sclerotherapy is commonly used to treat varicose veins.

Preferred sclerosing agents include mepacrine (quinacrine), chloroquine, polidocanol and quinine. Preferred sclerosing agents are agents that also provide a local anaesthetic effect, such as mepacrine, chloroquine and polidocanol.

Sclerosing agents that are acridines, such as mepacrine (quinacrine), are particularly preferred active agents.

The inventors have found that disbudding of an animal can be performed by administering one or more p53 activating agents to a horn bud.

Any suitable p53 activating agent or agents can be used. Suitable p53 activating agents that can be used in the invention are known to a skilled person, particularly for potential use in the treatment of cancer. The protein p53, also known as tumour protein p53 and tumour suppressor p53, functions as a tumour suppressor in cells. Agents that result in activation of the p53 protein are known as p53 activating agents.

Preferred p53 activating agents are small molecules, including mepacrine and chloroquine.

p53 activating agents that are acridines, such as mepacrine (quinacrine), are particularly preferred active agents.

Particularly preferred p53 activating agents provide a local anaesthetic effect, such as mepacrine, and chloroquine.

The inventors have found that disbudding of an animal can be performed by administering one or more antiprotozoal agents to a horn bud.

Any suitable antiprotozoal agent or agents can be used. Many antiprotozoal agents are known in the art. For instance, suitable antiprotozoal agents can include antimalarial agents. Techniques are known in the art for identifying and evaluating new potential antiprotozoal agents. Preferred antiprotozoal agents include mepacrine, chloroquine and quinine.

Antiprotozoal agents that are acridines, such as mepacrine (quinacrine), are particularly preferred agents.

Particularly preferred antiprotozoal agents are agents that also provide a local anaesthetic effect, such as mepacrine, and chloroquine.

The inventors have found that disbudding of an animal can be performed by administering one or more apoptosis inducing agents to a horn bud.

Any suitable apoptosis inducing agent or agents can be used. Suitable apoptosis inducing agents that can be used in the invention are known to a skilled person. Preferred apoptosis inducing agents include mepacrine and cinchocaine.

Apoptosis inducing agents that are acridines, such as mepacrine (quinacrine), are particularly preferred active agents. Particularly preferred apoptosis inducing agents provide a local anaesthetic effect, such as mepacrine, and cinchocaine.

The inventors have found that disbudding of an animal can be performed by administering one or more anaesthetic agents to a horn bud.

Any suitable anaesthetic agent or agents can be used. Suitable anaesthetic agents include an amide-, ester- or non-ionic surfactant-anaesthetic agent. Suitable anaesthetic agents include: lignocaine, chloroprocaine, mepivacaine, bupivacaine, articaine, etidocaine, levobupivacaine, tetracaine, prilocaine, benzocaine, ropivacaine, cocaine, hydroxyprocaine, hexocaine, dibucaine, perralcaine, and procaine, and pharmaceutically acceptable acids, bases, and salts thereof (including acidic salts). In some embodiments, the anaesthetic agent is present in the form of an acidic salt or an acidic solution. (Local anesthetic solutions typically have an acidic pH to maximize their water solubility.) Suitable anaesthetic agents include mepacrine (quinacrine), polidocanol, cinchocaine, chloroquine and quinine.

Chloroquine diphosphate can be administered, for example.

Preferably, the cytotoxic agent is mepacrine, polidocanol, cinchocaine, chloroquine or quinine. In addition to these being cytotoxic, these agents also provide a local anaesthetic effect.

The animal can be any type of animal that can develop true horns from horn buds. Such animals include, but are not limited to, bovine animals (cow or bull), goats, sheep, bison, African buffalo, gazelles, muskoxen and antelope. Preferably, the juvenile animal is a calf or a kid.

Any suitable quantity of cytotoxic agent or agents can be used, provided that it is cytotoxic to horn bud cells. The quantity administered depends on the potency of the cytotoxic agent/s, and the size or volume of the horn bud area being treated. The quantity of cytotoxic agent administered to horn bud cells is preferably in the range of approximately 5 to 1000 mg, including all numerical values between 5 and 1000. More preferably, the quantity of cytotoxic agent administered to horn bud cells is in the range of approximately 20 to 400 mg, including all numerical values between 20 to 400, including 20, 21, 22 etc.

For mepacrine, in some embodiments, at least approximately 100 mg (single injection or total for multiple injections) is administered. For mepacrine, preferably approximately 100 to 300 mg, preferably approximately 100 to 150 mg, and more preferably approximately 100 mg is administered, including all numerical values between 100 and 300, and 100 and 150 (100, 101, 102 etc). Salts of mepacrine such as mepacrine dihydrochloride or mepacrine dihydrochloride dihydrate can be administered, for example.

Typically, the cytotoxic agent will be administered to the animal in the form of a composition, formulation or medicament, suitable for injection. A preferred composition, formulation or medicament is a liquid composition. Preferred liquid compositions, formulations or medicaments include solutions, suspensions, dispersions, emulsions, low viscosity gels, and other prolonged-release formulations.

A preferred composition (formulation or medicament) for administration is a liquid composition, suitable for injection. Compositions for injection can be prepared by dissolving or mixing the cytotoxic agent in a suitable veterinary acceptable vehicle, carrier, solvent or excipient etc. which can include further ingredients such as a solubiliser, acid, base, buffer salt, anti-oxidant, and/or preservative. The compositions can be sterilised, such as by heat, filtration or irradiation, or prepared aseptically. Compositions for injection may be prepared by methods and techniques known to persons skilled in the art.

The composition (formulation or medicament) can include one or more of the following types of veterinary acceptable ingredients: a vehicle; an aqueous or oily diluent; a carrier; an excipient; a base; a buffering agent; a pH adjusting agent; a suspending agent; a flocculating agent; a thickener; a viscosity building agent; a gelling agent; a solvent; a co-solvent; a solvent system; an emulsifier; a stabilizer; a dispersant; a solubilizer; a fragrance; a preservative; a surfactant; a detergent, a complexing agent; an acid; a base; an anti-oxidant; a wetting agent; a chelating agent; a reducing agent; a bulking agent; a protectant; a tonicity adjustor; and, a colorant.

Examples of tonicity adjustors used in liquid injections include electrolytes, dextrose, glycerol, sodium chloride, glycerin and mannitol.

Examples of preservatives used in liquid injections include antioxidants, antimicrobials and chelating agents, including ascorbic acid, acetylcysteine, sulfurous acid salts (bisulfite, metabisulfite), monothioglyercol, phenol, meta-cresol, benzyl alcohol, parabens (methyl, propyl, butyl), benzalkonium chloride, chlorobutanol, thiomersal, phenylmercuric salts, butylated hydroxytoluene (BHT), butylated hydroxyanisole (BHA), and alpha tocopherol.

Examples of solubilising agents used in liquid injections include surfactants, solvents and co-solvents, and include water, Tween, polysorbate, non-ionic surfactant, polyoxyethylene sorbitan monooleate (Tween 80), EDTA, sorbitan monooleate polyoxyethylene sorbitan monolaurate (Tween 20), lecithin, polyoxyethylene copolymers (pluronics), propylene glycol, glycerin, ethanol, polyethylene glycol (300 and 400), sorbitol, dimethylacetamide and cremophor EL.

Examples of complexing and dispersing agents used in liquid injections include cyclodextrins and modified cyclodextrins such as hydroxypropyl-b-cyclodextrin and sulfobutylether-b-cyclodextrin.

Examples of buffering agents used in liquid injections include phosphate, citrate, acetate, lactate and tartrate buffers.

Suspensions, including aqueous or oily suspensions, may provide more prolonged release of the cytotoxic agent from the injection site than a comparable solution. Examples of excipients used in suspensions include flocculating/suspending agents, viscosity building agents, wetting agents, solvent systems, preservatives, anti-oxidants, chelating agents, buffering agents, and tonicity adjusting agents. Examples of excipients or oily suspending agents used in oily suspensions include oils, preferably vegetable oils, such as castor, cottonseed, soybean, ethyl oleate, olive, peanut, sesame, sunflower, soybean and safflower oil.

Examples of flocculating/suspending agents include electrolytes, surfactant and hydrophilic colloids, including potassium/sodium chloride, potassium/sodium citrate, and potassium/sodium acetate.

Examples of viscosity building agents include sodium carboxymethyl cellulose, acacia, gelatin, methyl cellulose, and polyvinyl pyrrolidone.

Examples of wetting agents include glycerin, alcohol, propylene glycol, lecithin, polysorbate 20, polysorbate 80, pluronic F-68, sorbitan trioleate.

Examples of solvents include water, ethanol, glycerin, propylene glycol, n-lactamide, and polyethylene glycol (PEG).

In some embodiments the composition (formulation or medicament) is in the form of a low viscosity, injectable gel which gels rapidly at the injection site to retain the cytotoxic agent for a suitable period of time. In this way a relatively larger quantity of cytotoxic agent can be delivered to the horn bud cells (horn bud tissue).

The composition can be made to gel in situ in any suitable way. In some embodiments, the composition comprises one or more gelling agents. In some embodiments, the composition comprises one or more solvents or a solvent system. In some embodiments, the composition comprises one or more surfactants. In some embodiments, the composition comprises Sucrose Acetate Isobutyrate (SAIB) and at least one type of solvent. In some embodiments, the composition comprises Sucrose Acetate Isobutyrate (SAIB), at least one type of solvent, and at least one type of surfactant. Once a composition containing SAIB formulation and solvent/s (and optionally surfactant/s) is injected, the solvent/s can diffuse out leaving a matrix that is both adhesive and viscous. The matrix can retain the cytotoxic agent at the horn bud cells for an effective period of time, rather than the cytotoxic agent dispersing almost immediately.

The SAIB may have a molecular weight of 846.9 (CAS number 27216-37-1 or 126-13-6).

In some embodiments, the composition comprises approximately 30% to 90% w/v SAIB, preferably approximately 40% to 85% w/v SAIB, more preferably approximately 70% to 80% w/v SAIB, and even more preferably approximately 70% w/v SAIB. These ranges include all numerical values and sub-ranges between 30 and 90, 40 and 85, and 70 to 80, including 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 86, 87, 88, 89 and 90.

Any suitable solvent or solvents can be used provided that they produce a low viscosity, injectable gel. Preferred solvents include ethanol, diethylene glycol monoethyl ether (DEGEE) sold under the trade mark Transcutol™, N-methylpyrrolidone (NMP), triacetin, benzyl benzoate, miglyol, propylene carbonate, benzyl alcohol, ethyl lactate, glycofurol, 2-pyrrolidone, propylene glycol, acetone, methyl acetate, ethyl acetate, methyl ethyl ketone, dimethylformamide, dimethylsulfoxide, tetrahydrofuran, caprolactam, decylmethylsulfoxide, oleic acid, and 1-dodecyazacycloheptan-2-one. The solvent or solvents can be present in an amount of between approximately 0-50% w/v, preferably approximately 10-30% w/v. These ranges include all numerical values and sub-ranges between 0 to 50, and 10 and 30, including 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50.

Any suitable surfactant or surfactants can be used provided that they produce a low viscosity, injectable gel. Preferred surfactants include non-ionic surfactants, polysorbates, Tweens, polyoxyethylene (20) sorbitan monooleate (Tween 80), and polyoxyethylene (20) sorbitan monolaurate (Tween 20). The surfactant or surfactants can be present in an amount up to approximately 0.25, 0.5, 0.75, 1.0, 1.25, 1.5, 1.75, 2.0, 2.25, 2.5, 2.75 or 3.0% w/v, or between approximately 0-3% w/v, 0-2% w/v, 0-1% w/v, 1-3% w/v, or 1-2% w/v, for example. These ranges include all numerical values and sub-ranges between 0-3, 0-2, 0-1, 1-3, and 1-2.

The concentration of cytotoxic agent in a composition administered to an animal is preferably in the range of approximately 0.01% weight/volume (w/v) to approximately 100% w/V, depending on the potency of the cytotoxic agent, including all numerical values and sub-ranges between 0.01 and 100, including approximately 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 86, 87, 88, 89, 90, 91, 92, 93, 94, 95, 96, 97, 98, 99 and 100.

The volume of composition administered to a horn bud is preferably within the range of approximately 0.05 to approximately 10 mL, including all numerical values between 0.05 and 10. A particularly preferred range is from 0.1 to 2.0 mL, including all numerical values between 0.1 and 2.0. For example, a composition quantity of approximately 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL or 1.5 mL can be administered to a horn bud. Preferably up to approximately 0.5 mL, 0.6 mL, 0.7 mL, 0.8 mL, 0.9 mL, 1 mL, 1.25 mL, or 1.5 mL of composition is injected.

The cytotoxic agent can be administered to the juvenile animal in any suitable way. Preferably, the cytotoxic agent is administered by injection. In some embodiments a single injection is administered. In some embodiments, more than one injection is administered, such as 2, 3 or 4 injections over a suitable period of time.

Preferably an injector is used to carry out injection of (administration to) the animal. Preferably an injector suitable for single-handed use is used, so as to allow use of the other hand to steady the animal's head and locate the horn bud.

In some embodiments, the injector is suitable for providing multiple doses, such as an automatic vaccinator injector, a self-refilling injection syringe, or a multi-dose automatic syringe. One example of a suitable injector is the McLintock syringe, conventionally used for tuberculin testing, but can be configured to administer the required dosage volume.

Preferred embodiments of the invention are defined in the numbered paragraphs below:
1. A method of chemically disbudding a juvenile animal or inhibiting horn growth in a juvenile animal by administering to horn bud cells of the animal at least one cytotoxic agent, wherein the at least one cytotoxic agent comprises at least one sclerosing agent, p53 activating agent, antiprotozoal agent, apoptosis inducing agent, or local anaesthetic agent.
2. Use of at least one cytotoxic agent for chemically disbudding a juvenile animal or inhibiting horn growth in a juvenile animal, wherein the at least one cytotoxic agent comprises at least one sclerosing agent, p53 activating agent, antiprotozoal agent, apoptosis inducing agent, or local anaesthetic agent.
3. At least one cytotoxic agent for use or when used for chemically disbudding a juvenile animal or inhibiting horn growth in a juvenile animal, wherein the at least one cytotoxic agent comprises at least one sclerosing agent, p53 activating agent, antiprotozoal agent, apoptosis inducing agent, or local anaesthetic agent.
4. Use of at least one cytotoxic agent in the manufacture of a medicament for chemically disbudding a juvenile animal or inhibiting horn growth in a juvenile animal, wherein the at least one cytotoxic agent comprises at least one sclerosing agent, p53 activating agent, antiprotozoal agent, apoptosis inducing agent, or local anaesthetic agent.
5. A composition comprising at least one cytotoxic agent, formulated for administration to and for chemically disbudding a juvenile animal or inhibiting horn growth in a juvenile animal, wherein the at least one cytotoxic agent comprises at least one sclerosing agent, p53 activating agent, antiprotozoal agent, apoptosis inducing agent, or local anaesthetic agent.
6. An injector containing at least one cytotoxic agent for use or when used for juvenile animal disbudding or for inhibiting horn growth in a juvenile animal, wherein the injector is capable of administering the at least one cytotoxic agent to horn bud cells of a juvenile animal, wherein the at least one cytotoxic agent comprises at least one sclerosing agent, p53 activating agent, antiprotozoal agent, apoptosis inducing agent, or local anaesthetic agent.
7. A kitset for use or when used in a method of juvenile animal disbudding or inhibiting horn growth in a juvenile animal, wherein the kitset comprises: an injector capable of administering at least one cytotoxic agent to horn bud cells of a juvenile animal; and, the at least one cytotoxic agent in an amount to kill horn bud cells, wherein the at least one cytotoxic agent comprises at least one sclerosing agent, p53 activating agent, antiprotozoal agent, apoptosis inducing agent, or local anaesthetic agent.
8. The method of paragraph 1, the use of paragraph 2, the at least one cytotoxic agent for use or when used in accordance with paragraph 3, the use according to paragraph 4, the composition of paragraph 5, the injector of paragraph 6, or the kitset of paragraph 7, wherein the at least one cytotoxic agent is in the form of a free-acid, free-base, or salt.
9. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises at least one sclerosing agent.
10. The method of paragraph 9, the use of paragraph 9, the at least one cytotoxic agent for use or when used in accordance with paragraph 9, the use according to paragraph 9, the composition of paragraph 9, the injector of paragraph 9, or the kitset of paragraph 9, wherein:
   the at least one sclerosing agent comprises mepacrine (quinacrine), chloroquine, polidocanol or quinine;
   the at least one sclerosing agent comprises an acridine, preferably mepacrine; or
   the at least one sclerosing agent is mepacrine.
11. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises at least one p53 activating agent.
12. The method of paragraph 11, the use of paragraph 11, the at least one cytotoxic agent for use or when used in accordance with paragraph 11, the use according to paragraph 11, the composition of paragraph 11, the injector of paragraph 11, or the kitset of paragraph 11, wherein:
   the at least one p53 activating agent comprises mepacrine or chloroquine;
   the at least one p53 activating agent comprises an acridine, preferably mepacrine; or
   the at least one p53 activating agent comprises mepacrine.
13. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises at least one antiprotozoal agent.
14. The method of paragraph 13, the use of paragraph 13, the at least one cytotoxic agent for use or when used in accordance with paragraph 13, the use according to paragraph 13, the composition of paragraph 13, the injector of paragraph 13, or the kitset of paragraph 13, wherein:
   the at least one antiprotozoal agent comprises at least one antimalarial agent;
   the at least one antiprotozoal agent comprises mepacrine, chloroquine or quinine;
   the at least one antiprotozoal agent comprises an acridine, preferably mepacrine; or
   the at least one antiprotozoal agent comprises mepacrine.
15. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises at least one apoptosis inducing agent.
16. The method of paragraph 15, the use of paragraph 15, the at least one cytotoxic agent for use or when used in accordance with paragraph 15, the use according to paragraph 15, the composition of paragraph 15, the injector of paragraph 15, or the kitset of paragraph 15, wherein:
   the at least one apoptosis inducing agent comprises mepacrine or cinchocaine;
   the at least one apoptosis inducing agent comprises an acridine, preferably mepacrine; or
   the at least one apoptosis inducing agent comprises mepacrine.
17. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises at least one anaesthetic agent.
18. The method of paragraph 17, the use of paragraph 17, the at least one cytotoxic agent for use or when used in accordance with paragraph 17, the use according to paragraph 17, the composition of paragraph 17, the injector of paragraph 17, or the kitset of paragraph 17, wherein:
   the at least one anaesthetic agent comprises an amide-, ester- or non-ionic surfactant-anaesthetic agent;
   the at least one anaesthetic agent comprises lignocaine, chloroprocaine, mepivacaine, bupivacaine, articaine, etidocaine, levobupivacaine, tetracaine, prilocaine, benzocaine, ropivacaine, cocaine, hydroxyprocaine, hexocaine, dibucaine, perralcaine, or procaine, or a pharmaceutically acceptable acid, base, or salt thereof;
   the at least one anaesthetic agent comprises mepacrine, polidocanol, cinchocaine, chloroquine or quinine;
   the at least one anaesthetic agent comprises an acridine, preferably mepacrine; or the at least one anaesthetic agent comprises mepacrine.
19. The method of paragraph 1 or 8, the use of paragraph 2 or 8, the at least one cytotoxic agent for use or when used in accordance with paragraph 3 or 8, the use according to paragraph 4 or 8, the composition of paragraph 5 or 8, the injector of paragraph 6 or 8, or the kitset of paragraph 7 or 8, wherein the at least one cytotoxic agent comprises mepacrine, polidocanol, cinchocaine, chloroquine or quinine.
20. The method of paragraph 19, the use of paragraph 19, the at least one cytotoxic agent for use or when used in accordance with paragraph 19, the use according to paragraph 19, the composition of paragraph 19, the injector of paragraph 19, or the kitset of paragraph 19, wherein:
   the amount of at least one cytotoxic agent administered or administrable comprises approximately 100 to 400 mg mepacrine as a free-base;
   the amount of at least one cytotoxic agent administered or administrable comprises approximately 100 to 400 mg mepacrine as a salt;
   the amount of at least one cytotoxic agent administered or administrable comprises approximately 100 to 400 mg mepacrine dihydrochloride;
   the amount of at least one cytotoxic agent administered or administrable comprises the equivalent of approximately 0.5 mL of polidocanol (95% to 100%);
   the amount of at least one cytotoxic agent administered or administrable comprises approximately 150 to 250 mg chloroquine as a free-base;
   the amount of at least one cytotoxic agent administered or administrable comprises approximately 150 to 250 mg chloroquine as a salt; or
   the amount of at least one cytotoxic agent administered or administrable comprises approximately 150 to 250 mg chloroquine diphosphate.
21. The method of any one of paragraphs 1 and 8 to 20, the use of any one of paragraphs 2 and 8 to 20, the at least one cytotoxic agent for use or when used in accordance with any one of paragraphs 3 and 8 to 20, the use according to any one of paragraphs 4 and 8 to 20, the composition of any one of paragraphs 5 and 8 to 20, the injector of any one of paragraphs 6 and 8 to 20, or the kitset of any one of paragraphs 7 and 8 to 20, wherein the at least one cytotoxic agent is administered or administrable to the juvenile animal by way of one or more injections, preferably as a single injection.
22. The method of any one of paragraphs 1 and 8 to 21, the use of any one of paragraphs 2 and 8 to 21, the at least one cytotoxic agent for use or when used in accordance with any one of paragraphs 3 and 8 to 21, the use according to any one of paragraphs 4 and 8 to 21, the composition of any one of paragraphs 5 and 8 to 21, the injector of any one of paragraphs 6 and 8 to 21, or the kitset of any one of paragraphs 7 and 8 to 21, wherein the at least one cytotoxic agent is administered or administrable to the juvenile animal in the form of a liquid composition capable of providing prolonged-release of the at least one cytotoxic agent.
23. The method of paragraph 22, the use of paragraph 22, the at least one cytotoxic agent for use or when used in accordance with paragraph 22, the use of paragraph 22, the composition of paragraph 22, the injector of paragraph 22, or the kitset of paragraph 22, wherein the liquid composition is in the form of a solution, suspension, dispersion, emulsion, or low viscosity gel.
24. The method of paragraph 23, the use of paragraph 23, the at least one cytotoxic agent for use or when used in accordance with paragraph 23, the use of paragraph 23, the composition of paragraph 23, the injector of paragraph 23, or the kitset of paragraph 23, wherein the liquid composition is in the form of a low viscosity, injectable gel formulated to gel rapidly at an injection site and prolong the release of the at least one cytotoxic agent from the gel.
25. The method of paragraph 24, the use of paragraph 24, the at least one cytotoxic agent for use or when used in accordance with paragraph 24, the use of paragraph 24, the composition of paragraph 24, the injector of paragraph 24, or the kitset of paragraph 24, wherein the low viscosity, injectable gel comprises one or more gelling agents, one or more solvents or a solvent system, and, optionally, one or more surfactants.
26. The method of paragraph 25, the use of paragraph 25, the at least one cytotoxic agent for use or when used in accordance with paragraph 25, the use of paragraph 25, the composition of paragraph 25, the injector of paragraph 25, or the kitset of paragraph 25, wherein the low viscosity, injectable gel comprises:
   Sucrose Acetate Isobutyrate (SAIB) as the one or more gelling agents and at least one type of solvent; or
   SAIB as the one or more gelling agents, at least one type of solvent, and at least one type of surfactant.
27. The method of paragraph 26, the use of paragraph 26, the at least one cytotoxic agent for use or when used in accordance with paragraph 26, the use of paragraph 26, the composition of paragraph 26, the injector of paragraph 26, or the kitset of paragraph 26, wherein the low viscosity, injectable gel comprises:
   approximately 30% to 90% w/v SAIB;
   approximately 40% to 85% w/v SAIB;
   approximately 70% to 80% w/v SAIB; or
   approximately 70% w/v SAIB.
28. The method of paragraph 26 or 27, the use of paragraph 26 or 27, the at least one cytotoxic agent for use or when used in accordance with paragraph 26 or 27, the use of paragraph 26 or 27, the composition of paragraph 26 or 27, the injector of paragraph 26 or 27, or the kitset of paragraph 26 or 27, wherein the low viscosity, injectable gel comprises:
   approximately 0-50% w/v of the at least one solvent; or
   approximately 15-30% w/v of the at least one solvent.
29. The method of paragraph 28, the use of paragraph 28, the at least one cytotoxic agent for use or when used in accordance with paragraph 28, the use of paragraph 28, the composition of paragraph 28, the injector of paragraph 28, or the kitset of paragraph 28, wherein the at least one solvent comprises one or more of ethanol, diethylene glycol monoethyl ether (DEGEE), N-methylpyrrolidone (NMP), triacetin, benzyl benzoate, miglyol, propylene carbonate, benzyl alcohol, ethyl lactate, glycofurol, 2-pyrrolidone, propylene glycol, acetone, methyl acetate, ethyl acetate, methyl ethyl ketone, dimethylformamide, dimethylsulfoxide, tetrahydrofuran, caprolactam, decylmethylsulfoxide, oleic acid, and 1-dodecyazacycloheptan-2-one.
30. The method of paragraph 26, 27, 28 or 29, the use of paragraph 26, 27, 28 or 29, the at least one cytotoxic agent for use or when used in accordance with paragraph 26, 27, 28 or 29, the use of paragraph 26, 27, 28 or 29, the composition of paragraph 26, 27, 28 or 29, the injector of paragraph 26, 27, 28 or 29, or the kitset of paragraph 26, 27, 28 or 29, wherein the low viscosity, injectable gel comprises:
   approximately 0-3% w/v of the at least one surfactant;
   approximately 0-2% w/v of the at least one surfactant;
   approximately 0-1% w/v of the at least one surfactant;
   approximately 1-3% w/v of the at least one surfactant; or
   approximately 1-2% w/v of the at least one surfactant.
31. The method of paragraph 30, the use of paragraph 30, the at least one cytotoxic agent for use or when used in accordance with paragraph 30, the use of paragraph 30, the composition of paragraph 30, the injector of paragraph 30, or the kitset of paragraph 30, wherein the at least one surfactant comprises one or more of a non-ionic surfactant, polysorbate, Tween, polyoxyethylene (20) sorbitan monooleate (Tween 80), or, polyoxyethylene (20) sorbitan monolaurate (Tween 20).
32. The method of paragraph 26, the use of paragraph 26, the at least one cytotoxic agent for use or when used in accordance with paragraph 26, the use of paragraph 26, the composition of paragraph 26, the injector of paragraph 26, or the kitset of paragraph 26, wherein the low viscosity, injectable gel comprises, all in approximate % w/v:
   10% w/v of the at least one cytotoxic agent, preferably mepacrine or a salt thereof that provides 10% w/v mepacrine, such as 11.8% w/v mepacrine dihydrochloride;
   40-85% w/v SAIB;
   0-2% w/v Tween 80;
   0-50% w/v Triacetin;
   0-50% w/v DEGEE; and
   ethanol to 100% by volume, wherein:
   the concentration of ethanol varies from 0-30% w/v; or
   in the absence of other solvents and Tween, and at 10% mepacrine base and a SAIB level of 80% w/v, the ethanol concentration is 20% w/v, assuming a weight per mL of the formulation is 1.1 g.
33. The method of paragraph 26, the use of paragraph 26, the at least one cytotoxic agent for use or when used in accordance with paragraph 26, the use of paragraph 26, the composition of paragraph 26, the injector of paragraph 26, or the kitset of paragraph 26, wherein the low viscosity, injectable gel comprises, all in approximate % w/v:
   20% w/v of the at least one cytotoxic agent, preferably mepacrine or a salt thereof that provides 20% w/v mepacrine, such as 23.6% w/v mepacrine dihydrochloride;
   40-85% w/v SAIB;
   0-2% w/v Tween 80;
   0-50% w/v Triacetin;
   0-50% w/v DEGEE; and
   ethanol to 100% by volume, wherein:
   the concentration of ethanol varies from 0-30% w/v; or
   in the absence of other solvents and Tween, and at 23.6% mepacrine dihydrochloride and a SAIB level of 85% w/v, the ethanol concentration is 1.4% w/v, assuming a weight per mL of 1.1 g, which may be too low in which case the maximum concentration of SAIB would have to be reduced.
34. The method of paragraph 26, the use of paragraph 26, the at least one cytotoxic agent for use or when used in accordance with paragraph 26, the use of paragraph 26, the composition of paragraph 26, the injector of paragraph 26, or the kitset of paragraph 26, wherein the low viscosity, injectable gel comprises, all in approximate % w/v:
   10% w/v of the at least one cytotoxic agent, preferably mepacrine or a salt thereof that provides 10% w/v mepacrine, such as 11.8% w/v mepacrine dihydrochloride;
   70% w/v SAIB;
   1% w/v Tween 80;
   5% w/v Triacetin;
   0% w/v DEGEE; and
   ethanol to 100% by volume, that is 22.2% w/v ethanol, assuming a weight per mL of 1.1
35. The method of paragraph 26, the use of paragraph 26, the at least one cytotoxic agent for use or when used in accordance with paragraph 26, the use of paragraph 26, the composition of paragraph 26, the injector of paragraph 26, or the kitset of paragraph 26, wherein the low viscosity, injectable gel comprises, all in approximate % w/v:
   20% w/v of the at least one cytotoxic agent, preferably mepacrine or a salt thereof that provides 20% w/v mepacrine, such as 23.6% w/v mepacrine dihydrochloride;
   70% w/v SAIB;
   1% w/v Tween 80;
   5% w/v Triacetin;
   0% w/v DEGEE; and
   ethanol to 100% by volume, that is 10.4% w/v ethanol, assuming a weight per mL of 1.1 g.
36. A composition formulated as a solution, suspension, dispersion, emulsion, low viscosity gel, or other prolonged-release liquid formulation, comprising at least one cytotoxic agent and one or more excipients, and being capable of providing prolonged-release of the at least one cytotoxic agent, wherein the at least one cytotoxic agent is one or more of a sclerosing agent, p53 activating agent, antiprotozoal agent, apoptosis inducing agent, and a local anaesthetic agent.
37. The composition of paragraph 36, formulated as an injectable oily solution or suspension, and comprising at least one oily excipient.
38. The composition of paragraph 37, comprising, all in approximate % w/v:
   10 to 15% w/v of at least one cytotoxic agent, preferably mepacrine or a salt thereof that provides 10 to 15% w/v mepacrine, such as 11.8 to 17.7% w/v mepacrine dihydrochloride; and
   an aqueous or oily suspending agent, preferably an oily suspending agent.
39. The composition of paragraph 36, formulated as a low viscosity, injectable gel.
40. The composition of paragraph 39, formulated to gel rapidly at an injection site of an animal and prolong the release of the at least one cytotoxic agent from the gel.
41. The composition of paragraph 39 or 40, comprising, all in approximate % w/v:
10 to 15% w/v of the at least one cytotoxic agent, preferably mepacrine or a salt thereof that provides 10 to 15% w/v mepacrine, such as 11.8 to 17.7% w/v mepacrine dihydrochloride.
42. The composition of paragraph 39, 40 or 41, wherein the low viscosity, injectable gel comprises one or more gelling agents, one or more solvents or a solvent system, and, optionally, one or more surfactants.
43. The composition of paragraph 42, wherein the low viscosity, injectable gel comprises:
   Sucrose Acetate Isobutyrate (SAIB) as the one or more gelling agents and at least one type of solvent; or
   SAIB as the one or more gelling agents, at least one type of solvent, and at least one type of surfactant.
44. The composition of paragraph 43, wherein the low viscosity, injectable gel comprises:
   approximately 30% to 90% w/v SAIB;
   approximately 40% to 85% w/v SAIB;
   approximately 70% to 80% w/v SAIB; or
   approximately 70% w/v SAIB.
45. The composition of paragraph 43 or 44, wherein the low viscosity, injectable gel comprises:
   approximately 0-50% w/v of the at least one solvent; or
   approximately 15-30% w/v of the at least one solvent.
46. The composition of paragraph 45, wherein the at least one solvent comprises one or more of ethanol, diethylene glycol monoethyl ether (DEGEE), N-methylpyrrolidone (NMP), triacetin, benzyl benzoate, miglyol, propylene carbonate, benzyl alcohol, ethyl lactate, glycofurol, 2-pyrrolidone, propylene glycol, acetone, methyl acetate, ethyl acetate, methyl ethyl ketone, dimethylformamide, dimethylsulfoxide, tetrahydrofuran, caprolactam, decylmethylsulfoxide, oleic acid, and 1-dodecyazacycloheptan-2-one.
47. The composition of paragraph 42, 43, 44, 45 or 46, wherein the low viscosity, injectable gel comprises:
   approximately 0-3% w/v of the at least one surfactant;
   approximately 0-2% w/v of the at least one surfactant;
   approximately 0-1% w/v of the at least one surfactant;
   approximately 1-3% w/v of the at least one surfactant; or
   approximately 1-2% w/v of the at least one surfactant.
48. The composition of paragraph 47, wherein the at least one surfactant comprises one or more of a non-ionic surfactant, polysorbate, Tween, polyoxyethylene (20) sorbitan monooleate (Tween 80), or, polyoxyethylene (20) sorbitan monolaurate (Tween 20).
49. The composition of paragraph 41, 42, 43, 44, 45, 46, 47 or 48, wherein the low viscosity, injectable gel comprises, all in approximate % w/V:
   10% w/v of the at least one cytotoxic agent, preferably mepacrine or a salt thereof that provides 10% w/v mepacrine, such as 11.8% w/v mepacrine dihydrochloride;
   40-85% w/v SAIB;
   0-2% w/v Tween 80;
   0-50% w/v Triacetin;
   0-50% w/v DEGEE; and
   ethanol to 100% by volume, wherein:
   the concentration of ethanol varies from 0-30% w/v; or
   in the absence of other solvents and Tween, and at a SAIB level of 80%, the ethanol concentration is 18.2% w/v, assuming a weight per mL of the formulation is 1.1 g.
50. The composition of paragraph 41, 42, 43, 44, 45, 46, 47 or 48, wherein the low viscosity, injectable gel comprises, all in approximate % w/v:
   20% w/v of the at least one cytotoxic agent, preferably mepacrine or a salt thereof that provides 20% w/v mepacrine, such as 23.6% w/v mepacrine dihydrochloride;
   40-85% w/v SAIB;
   0-2% w/v Tween 80;
   0-50% w/v Triacetin;
   0-50% w/v DEGEE; and
   ethanol to 100% by volume, wherein:
   the concentration of ethanol varies from 0-30% w/v; or
   in the absence of other solvents and Tween, and at a SAIB level of 85% w/v, the ethanol concentration is 1.4% w/v, assuming a weight per mL of 1.1 g, which may be too low in which case the maximum concentration of SAIB would have to be reduced.
51. The composition of paragraph 41, 42, 43, 44, 45, 46, 47 or 48, wherein the low viscosity, injectable gel comprises, all in approximate % w/v:
   10% w/v of the at least one cytotoxic agent, preferably mepacrine or a salt thereof that provides 10% w/v mepacrine, such as 11.8% w/v mepacrine dihydrochloride;
   70% w/v SAIB;
   1% w/v Tween 80;
   5% w/v Triacetin; 0% w/v DEGEE; and
   ethanol to 100% by volume, that is 22.2% w/v ethanol, assuming a weight per mL of 1.1 g.
52. The composition of paragraph 41, 42, 43, 44, 45, 46, 47 or 48, wherein the low viscosity, injectable gel comprises, all in approximate % w/v:
   20% w/v of the at least one cytotoxic agent, preferably mepacrine or a salt thereof that provides 20% w/v mepacrine, such as 23.6% w/v mepacrine dihydrochloride;
   70% w/v SAIB;
   1% w/v Tween 80;
   5% w/v Triacetin;
   0% w/v DEGEE; and
   ethanol to 100% by volume, that is 10.4% w/v ethanol, assuming a weight per mL of 1.1 g.
53. The composition of any one of paragraphs 36-52, wherein the at least one cytotoxic agent is mepacrine dihydrochloride.
54 The method of paragraph 1, the use of paragraph 2, the at least one cytotoxic agent for use or when used in accordance with paragraph 3, the use according to paragraph 4, the injector of paragraph 6, or the kitset of paragraph 7, wherein the at least one cytotoxic agent is formulated as the composition according to any one of paragraphs 36-53.

A composition, medicament or formulation as described herein, including in the Examples and Claims, including any one of the Tables shown herein.

### BRIEF DESCRIPTION OF THE FIGURES

Further aspects of the present invention will become apparent from the ensuing description (Examples) which is given by way of example only and with reference to the accompanying figures in which:

FIG. 1—Relating to Example 3. Chemical disbudding in calf using eugenol. Painless swelling at site of injection of eugenol.

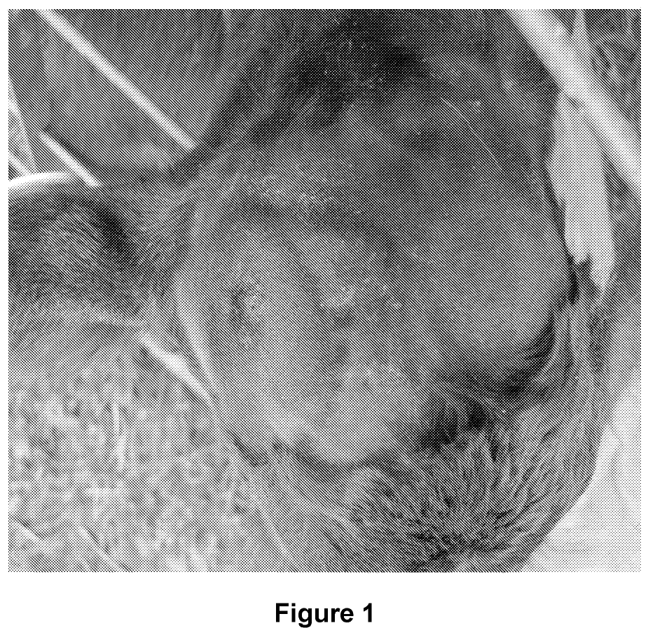

FIG. 2—Relating to Example 3. Representative images of different chemical disbudding calf treatments using: 1) eugenol; 2) mepacrine; 3) eugenol+aqueous mepacrine (20:80); and, 4) eugenol+aqueous mepacrine (80:20).

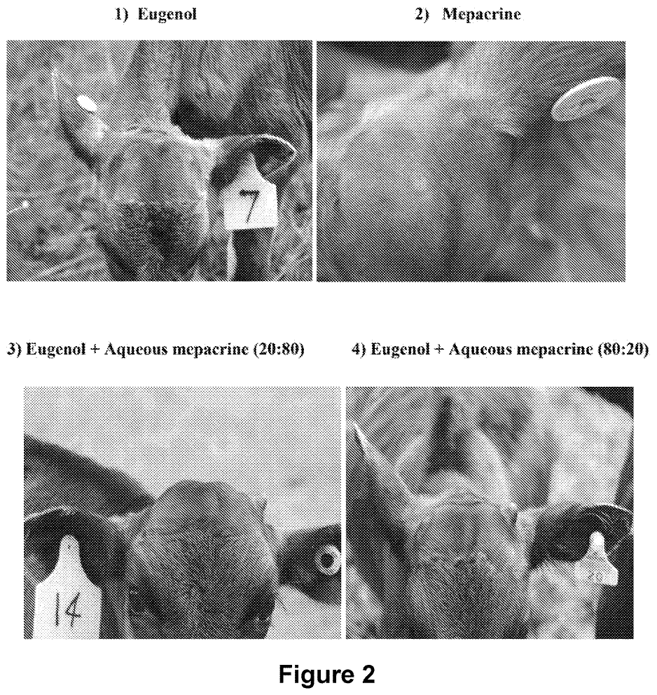

### EXAMPLES

### Example 1—Chemical Disbudding Using Mepacrine

A study was performed to test the disbudding efficacy from the administration to horn buds of mepacrine dihydrochloride, compared to a conventional hot iron disbudding method. The mepacrine dihydrochloride was administered as compositions comprising mepacrine dihydrochloride suspended in water. The compositions were prepared by dispersing mepacrine dihydrochloride powder into water for injection. Immediately before administration the compositions were shaken to re-suspend the mepacrine dihydrochloride.

The animals in the study (calves) were divided into multiple groups. Groups 1 to 4 were administered a single dose of 0.5 mL of mepacrine dihydrochloride composition, with each being administered a composition with a different concentration of mepacrine dihydrochloride, ranging from 50 mg/mL to 400 mg/mL.

Groups 5 to 8 were administered a divided dose of 0.5 mL of mepacrine dihydrochloride composition, with each being administered a composition with a different concentration of mepacrine dihydrochloride, ranging from 50 mg/mL to 400 mg/mL. Each divided dose comprised 5 doses of 0.1 mL administered around the horn bud.

The administration of the mepacrine dihydrochloride was performed by injecting the mepacrine dihydrochloride composition using a McLintock pre-set syringe configured to administer up to 0.5 mL. The single dose administration for groups 1 to 4 was performed by injecting the composition directly into the horn bud. The divided dose administration for groups 5 to 8 was performed by injecting the smaller doses to the area immediately around the horn bud. The animals in control group 9 were first treated with a 2% lignocaine injection as a cornual nerve block and then disbudded using a conventional hot-iron.

Each animal in the study had a disbudding procedure of either administration of mepacrine dihydrochloride or hot iron disbudding, applied to only the right horn bud. The left horn bud on each animal was left untreated as a control.

The animals in the study were bovine dairy calves, progressively enrolled in the study and treated at 7 days of age.

The study regime and results are shown in Table 1. The success rate is based on the proportion of treated horn buds that had no subsequent horn growth or presence of a scur. A horn scur is a distorted horn that is not attached to the skull. The mepacrine disbudding procedure was found to be most successful for the higher concentrations of mepacrine dihydrochloride. In particular, the administration of 0.5 mL of 200 mg/mL and 400 mg/L of mepacrine dihydrochloride had high success rates for both the single injection and the divided dose using multiple injections. The untreated horn bud on every animal developed into a horn.

The animals receiving the mepacrine disbudding treatment were observed as showing very little pain response. The animals receiving injections showed discomfort during the injections and no subsequent pain activity after completion of the injections.

TABLE 1
Mepacrine disbudding study

| | | | | Results (based on 90 days | | | |
|---|---|---|---|---|---|---|---|
| | | | Number of | observation) | | | |
| | | Conc. | animals | No horn | | Horn | Success |
| | Treatment | (mg/mL) | treated | growth | Scurs | growth | rate |
| 1 | Mepacrine | 400 | 10 | 8 | 2 | — | 80% |
| 2 | dihydrochloride | 200 | 10 | 8 | 1 | 1 | 80% |
| 3 | single injection (0.5 | 100 | 10 | 3 | — | 7 | 30% |
| 4 | mL) | 50 | 10 | 3 | — | 7 | 30% |
| 5 | Mepacrine | 400 | 10 | 7 | 2 | 1 | 70% |
| 6 | dihydrochloride | 200 | 10 | 9 | 1 | — | 90% |
| 7 | multiple injections | 100 | 10 | 5 | 3 | 2 | 50% |
| 8 | (5 × 0.1 mL) | 50 | 10 | — | 1 | 9 | 0% |
| 9 | Hot-iron disbudding | n/a | 10 | 8 | 2 | — | 80% |

There was a higher success rate in the higher concentration groups of 200 and 400 mg/mL compared to the lower concentration groups of 50 and 100 mg/mL. There was no noticeable advantage in splitting the dose into multiple injections.

The administration procedure of disbudding by administering mepacrine was less than 1 minute for each animal, compared to 5-10 minutes for the animals disbudded with the hot-iron. The animals treated with mepacrine experienced very little discomfort during administration and showed no pain reactions/behaviour.

### Example 2—Chemical Disbudding Using Chloroquine Diphosphate and Polidocanol

A study on calves was performed to test the disbudding efficacy from the administration to horn buds of chloroquine diphosphate and polidocanol.

The chloroquine diphosphate was administered as compositions comprising chloroquine diphosphate suspended in water. The compositions were prepared by dispersing chloroquine diphosphate powder into water for injection. Immediately before administration the compositions were shaken to re-suspend the chloroquine diphosphate.

The polidocanol was administered either as undiluted polidocanol when administered at 100% concentration, or as a diluted solution with water for the 90%, 50% and 10% concentration groups.

The animals in the study were divided into multiple groups. Groups 1 and 2 were administered saline solution (0.9% sodium chloride) administered as a single dose for group 1, and as divided doses for group 2. Each divided dose comprised 5 doses of 0.1 mL administered around the horn bud.

Groups 3 and 4 were administered chloroquine diphosphate at either 100 mg/ml or 300 mg/mL, and groups 5 to 8 were administered polidocanol are different concentrations ranging from 100% to 10% w/v.

The saline control groups resulted in no alteration to normal horn growth. The chloroquine diphosphate showed some success at a concentration of 300 mg/mL with half the animals having no horn growth at all, and the other have having only scurs forming. Polidocanol was successful at 100% concentration and 10% concentration, and less successful at the other concentrations. It was observed that the higher concentration compositions were highly viscous, and the lack of success may have been due to inadequate administration into the horn buds—eg. when at 90% concentration.

TABLE 2
Chloroquine and polidocanol disbudding study

| | | | | Results (based on 90 days | | | |
|---|---|---|---|---|---|---|---|
| | | | Number of | observation) | | | |
| | | | animals | No horn | | Horn | Success |
| | Treatment | Conc. | treated | growth | Scurs | growth | rate |
| 1 | Saline solution | 9 mg/mL | 4 | — | | 4 | 0% |
| | single injection | | | | | | |
| | (0.5 mL) | | | | | | |
| 2 | Saline solution | 9 mg/mL | 4 | — | — | 4 | 0% |
| | multiple injections | | | | | | |
| | (5 × 0.5 mL) | | | | | | |
| 3 | Chloroquine | 300 mg/ml | 4 | 2 | 2 | — | 50% |
| 4 | diphosphate | 100 mg/mL | 3 | — | 1 | 2 | 0% |
| | single injection | | | | | | |
| | (0.5 mL) | | | | | | |
| 5 | Polidocanol (0.5 | 100% | 1 | 1 | — | — | 100% |
| 6 | mL) | 90% | 4 | 1 | 2 | 1 | 25% |
| 7 | | 50% | 4 | — | 1 | 3 | 0% |
| 8 | | 10% | 1 | 1 | — | — | |

### Example 3—Chemical Disbudding Using Mepacrine and Eugenol

### Methodology

Sixteen calves (4 to 6 days old) obtained from commercial dairy farms were used in this study. The calves were randomly assigned to receive one of the four formulations (Table 3). Animals were gently restrained and the hair around the horn bud region was clipped. The formulations (0.3 to 0.5 mL) were then injected subcutaneously under the right horn bud or around the right horn bud using a McLintock syringe and needle (22G 11/64″, 0.71*4.3 mm) which delivered 0.1 mL on each injection. The left horn bud acted as control. The details of the treatment are given in the below Table 3. Animals were observed for pain related behaviors during and following the injections. The differences in the growth rate of the right and left horn buds were determined by measuring the height of the horn buds at regular intervals. Photos of the horn buds were taken at the same time. Calves will be euthanized after 4 months by intravenous injection of pentobarbitone sodium (100 mg/kg bodyweight) for gross pathology and histopathology of horn buds.

TABLE 3
Details of the treatment

| | Number of animals | |
|---|---|---|
| Formulations | Under the horn buds | Around the horn buds |
| Aqueous Mepacrine (400 | 5 (1 animal died) | 1 |
| mg/mL) | | |
| Eugenol (99%) | 3 | 2 |
| Eugenol + aqueous | 2 | — |
| Mepacrine (20:80) | | |
| Eugenol + aqueous | 3 | 1 |
| Mepacrine (80:20) | | |

Eugenol-Sigma Aldrich (Lot #STBG9481); Mepacrine or Quinacrine dihydrochloride-Sigma Aldrich (Lot #BCBR3327)

### Results

Minimal resistance was exhibited by the calves during the injection of the formulations. Each injection took less than a second and the total time for injection after restraining the calf was less than a minute. No pain related behavioral signs such as head shaking or head scratching were observed in any of the animals following the injection of the formulations. Swelling around the horn buds was observed following the injection of eugenol and eugenol+aqueous mepacrine (80:20). The swelling was painless to touch and resolved after few days. In addition, scars and depression were seen at the site of injection in the animals that received eugenol and eugenol+mepacrine (80:20). No such adverse effects were observed in the other two groups. The growth rates of horn buds are presented in Table 4.

TABLE 4
Growth rates of horn buds following the injection
of formulations on the right horn bud

| | | Horn growth rate (height | | | |
|---|---|---|---|---|---|
| | | of horn buds on 80th day | | | |
| Treat- | | minus height on 40th day) | | | |
| ments | Animal ID | Right horn bud | | Left horn bud | |
| Aqueous | Calf 1 (0.4 mL around | 7 mm | (18-11) | 10 mm | (26-16) |
| mepacrine | the horn bud) | | | | |
| (400 | Calf 9 (0.3 mL under | 0 mm | (9-9) | 9 mm | (22-13) |
| mg/mL) | the horn bud) | | | | |
| | Calf 10 (0.3 mL under | 0 mm | (6-6) | 10 mm | (24-14) |
| | the horn bud) | | | | |
| | Calf 11 (0.3 mL under | 0 mm | (7-7) | 9 mm | (24-15) |
| | the horn bud) | | | | |
| | Calf 12 (0.3 mL under | 0 mm | (7-7) | 8 mm | (16-8) |
| | the horn bud) | | | | |
| | Calf 16* (0.5 mL under | Died due to diarrhea | | | |
| | the horn bud) | | | | |
| Eugenol | Calf 2 (0.4 mL around | 2mm | (19-17) | 15 mm | (30-15) |
| | the horn bud) | | | | |
| | Calf 4 (0.5 mL around | 2 mm | (10-8) | 14 mm | (30-16) |
| | the horn bud) | | | | |
| | Calf 468 (0.3 mL under | 0 mm | (8-8) | 13 mm | (30-17) |
| | the horn bud) | | | | |
| | Calf 7 (0.2 mL under | 0 mm | (5-5) | 5 mm | (11 -6) |
| | the horn bud) | | | | |
| | Calf 8 (0.5 mL under | 8 mm | (22-14) | 8 mm | (30-22) |
| | the horn bud) | | | | |
| Eugenol + | Calf 13 (0.3 mL under | 0 mm | (9-9) | 8 mm | (16-28) |
| Aqueous | the horn bud) | | | | |
| mepacrine | Calf 14 (0.3 mL under | 0 mm | (7-7) | 10 mm | (27-17) |
| (20:80) | the horn bud) | | | | |
| Eugenol + | Calf 3 (0.4 mL around | 8 mm | (19-17) | 3 mm | (30-27) |
| Aqueous | the horn bud) | | | | |
| mepacrine | Calf 5 (0.4 mL around | 0 mm | (8-8) | 15 mm | (26-11) |
| (80:20) | the horn bud) | | | | |
| | Calf 15 (0.3 mL under | 0 mm | (4-4) | 8 mm | (22-14) |
| | the horn bud) | | | | |

### SUMMARY

TABLE 5
Subjective assessment of efficacy

| Treatments | N | Effective | Ineffective |
|---|---|---|---|
| Aqueous mepacrine | 5 | 4 | 1 (around the horn bud) |
| (400 mg/mL) | | | |
| Eugenol | 5 | 3 | 2 (1 - around the horn bud) |
| Eugenol + Aqueous | 2 | 2 | |
| mepacrine (20:80) | | | |
| Eugenol + Aqueous | 3 | 2 | 1 (around the horn bud) |
| mepacrine (80:20) | | | |

### Findings for Example 3

The disbudding techniques of this Example appear to be simple, fast, effective and eliminate the need for post-operative pain relief.

Main findings:
1) Injection of the formulations using a McLintock syringe and needle was fast, simple and caused minimal pain.
2) All the formulations appear to prevent the growth when injected under the horn buds.
3) Injection around the horn buds reduced the growth but failed to completely prevent growth in most of the animals.
4) Eugenol and its combination (eugenol+aqueous mepacrine (80:20)) produced slight swelling and scars at the site of injection. No such effects were seen following the injection of aqueous mepacrine or eugenol+aqueous mepacrine (20:80).
5) Absence of pain related behavioral signs after injection may mean that post-operative pain relief is not required if these formulations are used.

### General Conclusion

The present inventors have found that mepacrine, polidocanol and chloroquine function as effective chemical disbudding agents, and thereby overcome or reduce many of the disadvantages of previously known disbudding procedures.

Injection of these chemical disbudding agents seems to be well tolerated by animals.

Administration of these chemical disbudding agents is safer and easier compared with conventional dehorning techniques.

Single injection of these chemical disbudding agents can dehorn animals.

These chemical disbudding agents are targeted to horn bud cells/horn buds/horn bud tissues, and so cause much less collateral damage to surrounding tissues, as compared with some conventional dehorning techniques.

These chemical disbudding agents provide a local anaesthetic effect, thereby negating the need for post-operative pain relief.

Unlike eugenol, these chemical disbudding agents do not produce swelling at the site of injection.

Unlike eugenol, these chemical disbudding agents do not produce open wounds nor scars at the site of injection, thereby reducing the chance of infection.

### Example 4—Low Viscosity, Injectable Gel Formulations: SAIB+Cytotoxic Agent

It is believed that a composition providing prolonged release of the cytotoxic agent from the injection site may provide equal or superior disbudding results. It is believed that a lower amount of cytotoxic agent could be used in an injectable prolonged release composition as compared to the ones tested. It is envisaged that the administered composition could be in the form of a low viscosity, injectable gel (solution or suspension) which gels rapidly at the injection site to retain the cytotoxic agent for a suitable (therapeutically effective) period of time. In this way a comparatively larger quantity of cytotoxic agent can be delivered to the horn bud cells (horn bud tissue). It is believed that a composition containing the cytotoxic agent, Sucrose Acetate Isobutyrate (SAIB) and a solvent/s could achieve this. Once the composition containing SAIB formulation and solvent is injected, the solvent can diffuse out leaving a matrix that is both adhesive and viscous. The matrix can retain the cytotoxic agent at the horn bud cells for a period of time, rather than the cytotoxic agent dispersing immediately.

Preferred cytotoxic agent+SAIB formulations are described below.

### Ingredient Ranges

Although the solvents triacetin, Transcutol™ (diethylene glycol monoethyl ether) and ethanol are exemplified below, other solvents may be used instead or in addition to these. The inventors have worked on the principles that they will need 40-85% SAIB and ethanol, up to 30% to adjust the release rate of the cytotoxic agent.

TABLE 6
Cytotoxic agent + SAIB formulation ingredient ranges

| Ingredients | g | % w/v |
|---|---|---|
| Cytotoxic agent** | 0.1 | 10 |
| SAIB | 0.4-0.85 | 40-85 |
| Tween 80 | 0.0-0.02 | 0-2 |
| Triacetin | 0-0.5 | 0-50 |
| Transcutol ™ | 0-0.5 | 0-50 |
| Ethanol up to* | 1.0 mL | 100 |

** Mepacrine dihydrochloride is exemplified.

*Mass of ethanol to vary from 0 to 0.3 g, ie. 0-30% w/v. In the absence of other solvents and Tween, and at a SAIB level of 0.85 g (85%), the ethanol would be 0.15 g (15% w/v) assuming the formulation has a weight per mL of 1.1 g.

TABLE 7
Cytotoxic agent + SAIB formulation ingredient ranges

| Ingredients | g | % w/v |
|---|---|---|
| Cytotoxic agent** | 0.3 | 20 |
| SAIB | 0.6-1.275 | 40-85 |
| Tween 80 | 0.0-0.03 | 0-2 |
| Triacetin | 0-0.75 | 0-50 |
| Transcutol ™ | 0-0.75 | 0-50 |
| Ethanol up to* | 1.5 mL | 100 |

** Mepacrine dihydrochloride is exemplified.

*Mass of ethanol may vary from 0 to 0.45 g, ie. 0-30% w/v and in the absence of other solvents and Tween and at a SAIB level of 1.275 g (85%), the ethanol would be 0.075 g (5%) which may be too low in which case the maximum concentration of SAIB would have to be reduced.

TABLE 8
Preferred cytotoxic agent + SAIB formulation

| Ingredients | g | % w/v |
|---|---|---|
| Cytotoxic agent** | 0.1 | 10 |
| SAIB | 0.7 | 70 |
| Tween 80 | 0.01 | 1 |
| Triacetin | 0.05 | 5 |
| Transcutol ™ | 0 | 0 |
| Ethanol up to* | 1.0 mL | 100 (ie. approx. 24% w/v) |

** Mepacrine as the dihydrochloride is exemplified hence 0.118 g of mepacrine dihydrochloride

*Mass of ethanol required is 0.22 g or 22% w/v (assuming a weight per mL of 1.1 g).

TABLE 8
Preferred cytotoxic agent + SAIB formulation

| Ingredients | g | % w/v |
|---|---|---|
| Cytotoxic agent ** | 0.3 | 20 |
| SAIB | 0.975 | 65 |
| Tween 80 | 0.015 | 1 |
| Triacetin | 0.075 | 5 |
| Transcutol ™ | 0 | 0 |
| Ethanol up to* | 1.5 mL | 100 (ie. approx. 19% w/v) |

** Mepacrine as the dihydrochloride is exemplified hence 0.355 g of mepacrine dihydrochloride.

*Mass of ethanol required is 0.23 g or 15.3% w/v (assuming a weight per mL of the formulation of 1.1 g).

All estimates of the % w/v ethanol have assumed a weight per mL of the formulation of 1.1 g. The actual values will vary, for example, as the SAIB content is reduced the weight per mL is likely to be lower. Actual values would be determined experimentally using well-known methods.

Aspects of the present invention have been described by way of example only and it should be appreciated that modifications and additions may be made thereto without departing from the scope thereof as defined in the appended claims.

All references, including any patents or patent applications cited in this specification are hereby incorporated by reference. No admission is made that any reference constitutes prior art. It will be clearly understood that, although a number of prior art publications are referred to herein, this reference does not constitute an admission that any of these documents form part of the common general knowledge in the art in any country.

Throughout this specification, the word ‘comprise’, or variations thereof such as ‘comprises’ or ‘comprising’, will be understood to imply the inclusion of a stated element, integer or step, or group of elements integers or steps, but not the exclusion of any other element, integer or step, or group of elements, integers or steps.

Context permitting, the term ‘approximately’ or ‘about’ generally means up to 10%, plus or minus, of the referenced number, but this need not be the case.

1. A method of chemically disbudding a juvenile animal or inhibiting horn growth in a juvenile animal by administering to horn bud cells of the animal at least one cytotoxic agent, wherein the at least one cytotoxic agent is selected from the group consisting of a sclerosing agent, p53 activating agent, antiprotozoal agent, apoptosis inducing agent, and a local anaesthetic agent.

2.-54. (canceled)

55. The method of claim 1, wherein the at least one cytotoxic agent is selected from the group consisting of an acridine, mepacrine, chloroquine, polidocanol, and quinine.

56. The method of claim 55, wherein the at least one cytotoxic agent is administered to the juvenile animal in a single injection.

57. The method of claim 55, wherein the at least one cytotoxic agent is administered to the juvenile animal in multiple injections.

58. The method of claim 55, wherein the at least one cytotoxic agent is administered to the juvenile animal in the form of an injectable liquid composition.

59. The method of claim 58, wherein the injectable liquid composition is in a form selected from the group consisting of a solution, suspension, dispersion, emulsion, and low viscosity gel.

60. The method of claim 59, wherein the at least one cytotoxic agent is administered to the juvenile animal in the form of a low viscosity, injectable gel formulated to gel rapidly at an injection site and prolong the release of the at least one cytotoxic agent from the gel.

61. The method of claim 55, wherein the at least one cytotoxic agent is administered to the juvenile animal after mixing the at least one cytotoxic agent with a suitable veterinary acceptable vehicle, carrier, solvent or excipient.

62. The method of claim 55, wherein the at least one cytotoxic agent is an acridine.

63. The method of claim 55, wherein the at least one cytotoxic agent is mepacrine.

64. The method of claim 63, wherein the at least one cytotoxic agent is mepacrine salt.

65. The method of claim 55, wherein an amount of the at least one cytotoxic agent administered to the juvenile animal is selected from the group consisting of: approximately 100 to 400 mg mepacrine salt; approximately 100 to 400 mg mepacrine dihydrochloride; and, approximately 100 to 400 mg mepacrine dihydrochloride dihydrate.

66. A composition comprising at least one cytotoxic agent, formulated for administration by injection to and for chemically disbudding a juvenile animal or inhibiting horn growth in a juvenile animal, wherein the at least one cytotoxic agent is selected from the group consisting of a sclerosing agent, p53 activating agent, antiprotozoal agent, apoptosis inducing agent, and local anaesthetic agent.

67. The composition of claim 66, wherein the at least one cytotoxic agent is selected from the group consisting of an acridine, mepacrine, chloroquine, polidocanol, and quinine.

68. The composition of claim 66, wherein the composition is in a form selected from the group consisting of an injectable solution, suspension, dispersion, emulsion, and low viscosity gel.

69. The composition of claim 67, wherein the at least one cytotoxic agent comprises an acridine.

70. The composition of claim 67, wherein the at least one cytotoxic agent comprises mepacrine.

71. The composition of claim 70, wherein the at least one cytotoxic agent comprises mepacrine salt.

72. The composition of claim 71, formulated to administer by injection approximately 100 to 400 mg mepacrine salt.

73. The composition of claim 72, wherein the mepacrine salt is selected from the group consisting of mepacrine dihydrochloride and mepacrine dihydrochloride dihydrate.
